(19) United States
(12) Patent Application Publication
(10) Pub. No.: US 2005/0251102 A1
(43) Pub. Date: Nov. 10, 2005

(54) Catheter connection systems and methods

(76) Inventors: Michael Hegland, Mounds View, MN (US); James M. Olsen, Plymouth, MN (US); James G. Skakoon, St. Paul, MN (US)

Correspondence Address: MUETING, RAASCH & GEBHARDT, P.A., P.O. BOX 581415, MINNEAPOLIS, MN, 55458, US

(21) Appl. No.: 11/087,927

(22) Filed: Mar. 23, 2005

Related U.S. Application Data
(63) Continuation-in-part of application No. 10/950,727, filed on Sep. 27, 2004.
(63) Continuation-in-part of application No. PCT/US04/31518, filed on Sep. 27, 2004.
(60) Provisional application No. 60/506,594, filed on Sep. 26, 2003.
(60) Provisional application No. 60/506,594, filed on Sep. 26, 2003.

(51) Int. Cl. A61M 31/00

(52) U.S. Cl. 604/500; 604/533; 604/288.04

(57) ABSTRACT

Systems for coupling a catheter to a medical device port and methods for relieving stress on a catheter coupled to a medical device port. Exemplary systems may include both a seal attached at or near a proximal end of the catheter, and a tubular restraint. The restraint may include a first interlocking surface formed on the interior surface at or near the proximal end that engages a second interlocking surface associated with the port. The system may also include a locking sleeve to secure the restraint in the desired position. Exemplary methods may include: coupling the port to the proximal end of the catheter and associated seal; sliding the restraint over the seal; and engaging the first and second interlocking surfaces.

### RELATED APPLICATIONS

This application is a continuation-in-part of: U.S. patent application. Ser. No. 10/950,727, filed Sep. 27, 2004; and International Patent Application Serial No. PCT/US2004/031518, filed Sep. 27, 2004, both of which claim the benefit of U.S. Provisional Application No. 60/506,594, filed Sep. 26, 2003, all of which are incorporated herein by reference in their respective entireties.

### TECHNICAL FIELD

This application relates generally to medical devices and, more specifically, to catheter connection systems and methods for connecting medical tubing to implanted medical devices.

### BACKGROUND

Implantable infusion pump systems are know for delivering a therapeutic agent to a specific site within a body, particularly when the agent must be administered to the site in a tightly controlled, yet minute, dosage. Such pump systems typically contain a reservoir operably connected to a delivery catheter. The reservoir, which holds the therapeutic agent, may be disposed within a housing of the infusion pump.

The catheter is typically connected to a port or connector of the pump by one or more sutures. While effective, suture-attached catheters can disconnect from the pump between about 0.7% and about 1.4% of the time in clinical use. In addition, undesired cuts and punctures are a potential source of complications with suturing catheters to pumps, occurring at a rate of about 0.6% to about 6.2% in clinical use. Still further, use of sutures to secure catheters to pumps is subject to physician variability in suture tightness, e.g., some physicians may suture too tightly while others suture too loosely. Replacement of catheters that are secured via sutures may also be a source of concern, as caution is needed in removing the suture from the pump connector.

Additionally, certain systems for connecting a catheter to a port or connector of a medical device (e.g., pump) may use, in addition to or in place of sutures, connectors that apply a sustained compression force to the catheter to secure it in place relative to the port. Such systems, while they may be effective to hold the catheter securely with respect to the port, may, in some instances, place such stress on the catheter that the integrity of the catheter may be impaired and/or compromised. This is of particular concern when a connection system is used for long term implantation of a medical device, such as an implantable pump.

### BRIEF SUMMARY

Embodiments of the present invention provide connector or connection systems for coupling a catheter to a port of a medical device. These connection systems may provide a secure yet releasable connection between the catheter and the medical device port without the need for sutures. Embodiments of the present invention may further provide methods for coupling a catheter to a medical device, and to methods of relieving stress placed on a catheter once it is so coupled.

In one embodiment of the present invention, a connection system for coupling a catheter to a port of a medical device is provided. The system includes a seal attached at or near a proximal end of the catheter, wherein the seal has a flanged seal element defining an annular face that is substantially normal to a longitudinal axis of the catheter. A tubular restraint is also provided and configured to slide partially or completely over the seal. The restraint includes: a contact portion located on an interior surface of the restraint, wherein the contact portion is configured to abut the annular face of the flanged seal element; and a first interlocking surface formed on an interior surface at or near a proximal end of the restraint. The first interlocking surface is configured to engage a second interlocking surface associated with the port of the medial device, wherein engagement of the first interlocking surface with the second interlocking surface substantially secures the catheter relative to the port.

In another embodiment, a connection system for coupling medical tubing to a port of a medical device is provided wherein the system includes a catheter assembly. The catheter assembly includes the medical tubing and a seal attached at or near a proximal end of the medical tubing. The seal includes: a flanged seal element defining an annular face that is substantially normal to a longitudinal axis of the medical tubing; and a tapered sealing portion extending distally from the annular face of the flanged seal element. Also included is a tubular restraint configured to slide partially or completely over the seal. The restraint includes: a contact portion located on an interior surface of the restraint, the contact portion configured to abut the annular face of the flanged seal element; and a first interlocking surface formed on the interior surface at or near a proximal end of the restraint. The first interlocking surface is configured to engage a second interlocking surface associated with the port of the medical device, wherein engagement of the first interlocking surface with the second interlocking surface substantially secures the medical tubing relative to the port. A locking sleeve configured to surround the restraint is also provided with the system, wherein the locking sleeve is securable relative to the restraint.

In yet another embodiment, a method for relieving stress on a catheter coupled to a port of a medical device is provided, wherein the method includes coupling the port to a proximal end of the catheter. The proximal end of the catheter includes a flanged seal element defining an annular face substantially normal to a longitudinal axis of the catheter, wherein the flanged seal element defines an opening for receiving the port, the opening in fluid communication with a lumen of the catheter. The method also includes sliding a tubular restraint over the flanged seal element, wherein the restraint includes: a contact portion located on an interior surface of the restraint, the contact portion operable to abut the annular face of the flanged seal element; and a first interlocking surface located on the interior surface at or near a proximal end of the restraint. The method also includes engaging the first interlocking surface of the restraint with a second interlocking surface located on the port.

The above summary of the invention is not intended to describe each embodiment or every implementation of the present invention. Rather, a more complete understanding of the invention will become apparent and appreciated by reference to the following detailed description and claims in view of the accompanying drawings.

### BRIEF DESCRIPTION OF THE DRAWING

The advantages of the present invention will become more apparent by referring to the following detailed description and accompanying figures of the drawing, wherein:

FIGS. 1A-1C illustrate a catheter connection system in accordance with one embodiment of the invention where a catheter is connected to a port of a medical device, e.g., implantable infusion pump, by a sliding type connector, wherein: FIG. 1A shows the system components prior to connection; FIG. 1B shows the catheter partially connected but in an unlocked position; and FIG. 1C shows the system in a locked position;

FIGS. 2A and 2B illustrate embodiments of the invention similar to that shown in FIGS. 1A-1C, where a catheter is connected to the port by a sliding type connector system, wherein FIG. 2A shows the system in a locked position, and FIG. 2B shows a similar system also in a locked position;

FIG. 3 illustrates a cross section of a catheter connected to a pump port by a connection system in accordance with the embodiment of FIGS. 1A-1C;

FIG. 4 illustrates a cross section of the connection system of FIGS. 1A-1C with a restraint shown in an abutting relation with a seal of the catheter;

FIG. 5 illustrates another embodiment of the present invention wherein a topping mechanism is affixed to a catheter of the connection system to prevent embers of the connection system from sliding distally off the catheter;

FIGS. 6A and 6B illustrate yet another embodiment of a connection system in accordance with the present invention wherein a catheter is connected to a pump port by a sliding ring type connector, wherein FIG. 6A shows the system in an unlocked position, and FIG. 6B shows it in a locked position;

FIGS. 7A and 7B illustrate a connection system in accordance with another embodiment of the invention wherein a catheter is connected to a pump port by a snap type connector, wherein FIG. 7A shows the system in an unlocked position, and FIG. 7B shows it in a locked position;

FIGS. 8A and 8B illustrate yet another embodiment of a connection system of the present invention where a catheter is connected to a pump port by a snap ring type connector, wherein FIG. 8A shows the system in an unlocked position, and FIG. 8B shows it in a locked position;

FIG. 9 illustrates a further embodiment of a connection system of the present invention where a catheter is connected to a pump port by a sliding ring type connector; and

FIG. 10 illustrates a still further embodiment of a connection system of the present invention where a catheter is connected to a pump port by a screw on ring type connector.

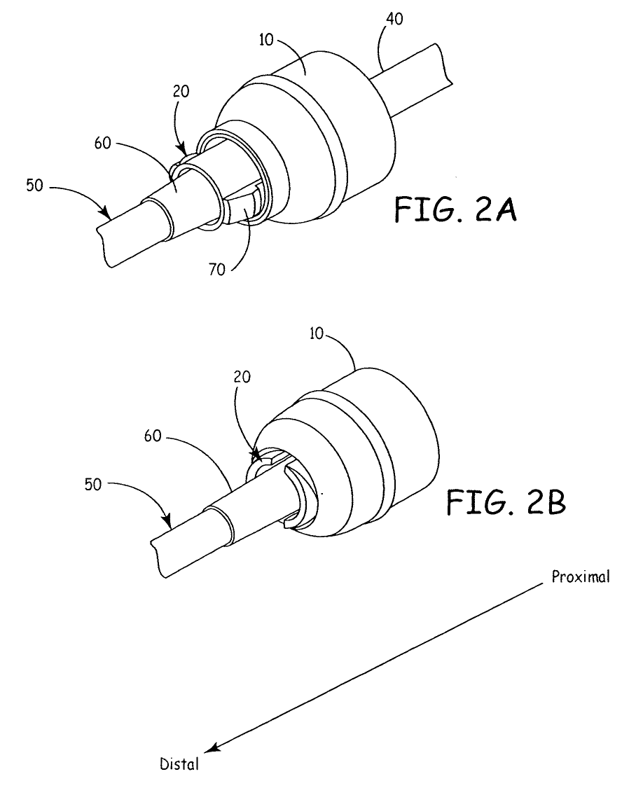

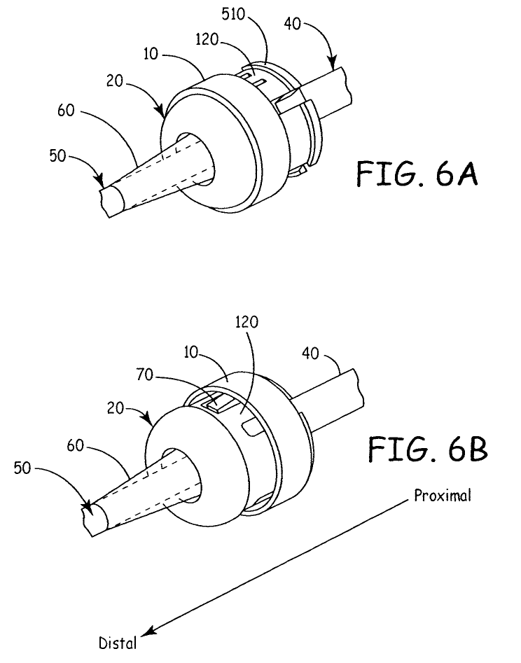

The drawings are not necessarily rendered to scale. Like numbers refer to like parts or steps throughout the drawings. However, it should be understood that use of like reference numbers are for convenience and should not be construed as limiting. For example, the use of the number “10” to refer to “locking sleeve” in both FIGS. 6A and 2A does not indicate that the locking sleeve of FIG. 6A must take the form of the locking sleeve shown in FIG. 2A, as is clearly depicted in the figures.

### DETAILED DESCRIPTION

In the following descriptions, reference is made to the accompanying figures of the drawing that form a part hereof, and in which are shown by way of illustration several specific embodiments of the invention. It is to be understood that other embodiments of the present invention are contemplated and may be made without departing from the scope of the present invention. The following detailed description, therefore, is not to be taken in a limiting sense.

I. Overview

Connection systems in accordance with embodiments of the present invention may include a restraint, e.g., a restraint sleeve, configured to engage both a seal that is attached (or otherwise formed with) an end of a catheter, and an interlocking surface associated with a port of a medical device (such as an implantable infusion pump) as further discussed below. The connection system may provide a well-sealed connection with little or no compression, particularly little or no sustained compression, exerted by the system on the catheter itself. An optional locking member or sleeve may also be provided to assist in maintaining the restraint relative to the port and the seal.

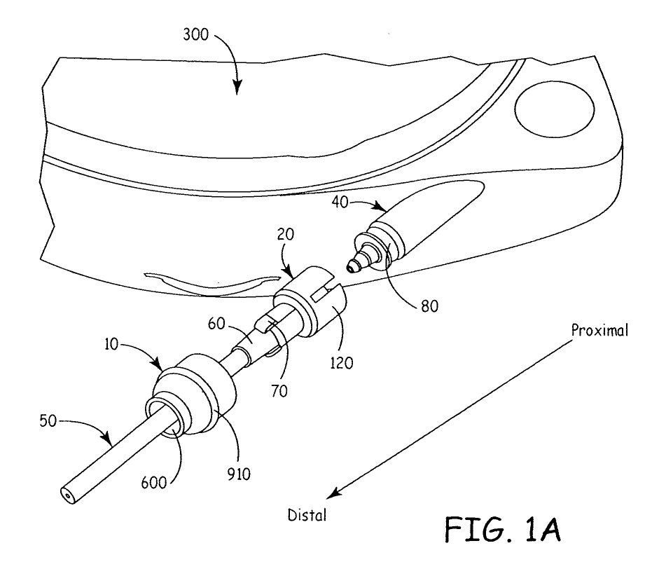

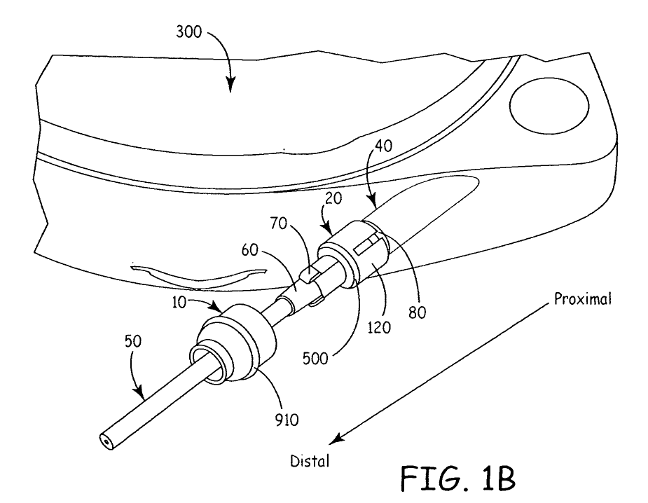

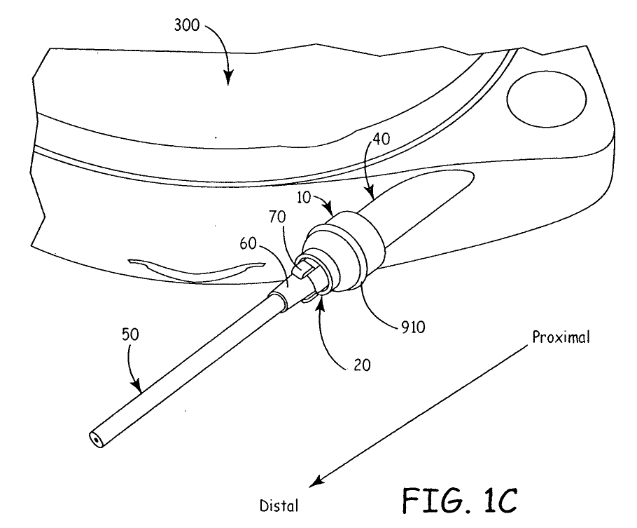

For clarity, an embodiment of the present invention is described below primarily with reference to FIGS. 1A-1C, 2A-2B, 3, and 4. However, other alternative embodiments are described and illustrated herein, and these and other embodiments are certainly possible without departing from the scope of the invention.

Moreover, this description includes headings and subheadings for organizational purposes only. The particular headings/subheadings are not intended to limit in any way the interpretation of the embodiments described therein, i.e., alternative embodiments may certainly be found elsewhere in the specification. Thus, the specification is to be viewed and understood for the entirety of its teachings.

A. Restraint and Locking Member

In the embodiment of the invention illustrated in FIGS. 1A-1C, a connection system for securing a catheter 50 to a port 40 of a medical device (e.g., an implantable infusion pump 300) is provided. The illustrated system may include a tubular restraint 20 (see FIGS. 1A-1C and 2A-2B) that is slidable along the catheter 50 and is configured to secure the catheter relative to the port 40 of the pump 300 as further described below. The connection system may further include an optional locking member, e.g., locking sleeve 10, to assist in maintaining the restraint 20 relative to the pump 300 and the catheter 50. The locking sleeve 10, like the restraint 20, is movable, e.g., slidable, along the catheter 50 as shown in the figures. FIG. 2A shows a system wherein the locking sleeve 10 is in a locked position with respect to tabs 70 (discussed in greater detail below). FIG. 2B shows a similar system, wherein, with respect to the embodiment shown in FIG. 2A, the tabs 70 are of a different shape, and the restraint further includes notched portions at the distal end.

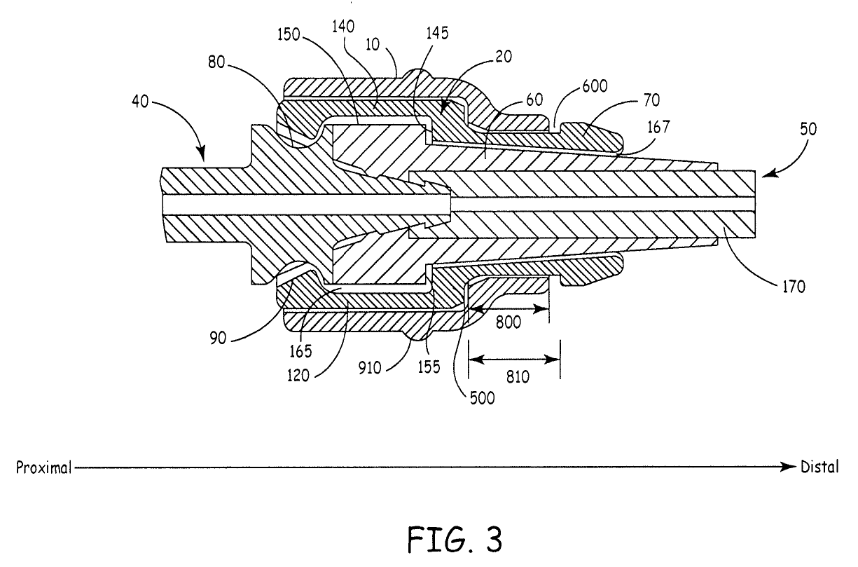

The restraint 20 may include one or more, e.g., a plurality of, extension members 120. The extension members 120 may form a first interlocking portion or surface 90 configured to engage a second interlocking portion or surface 80 of the port 40, as further described below and shown, for example, in FIG. 3.

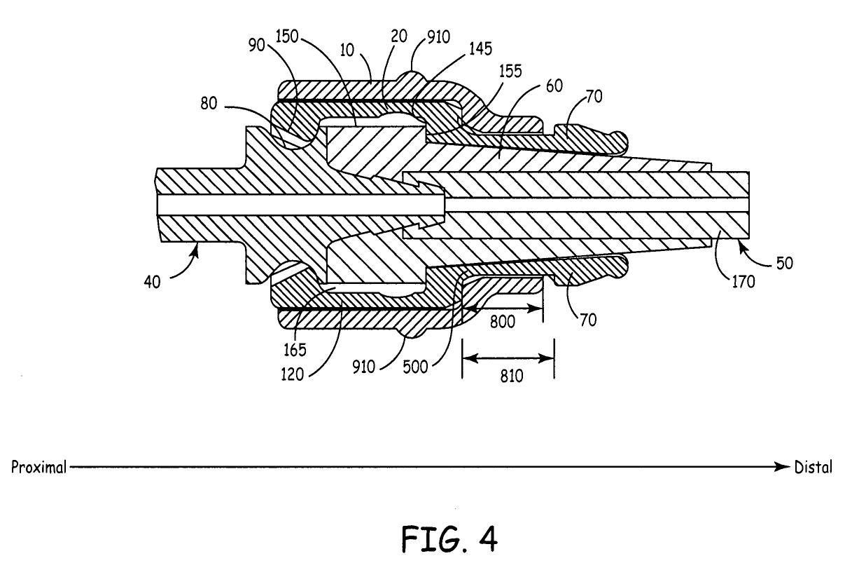

As FIGS. 1A-1C further illustrate, a seal 60 may also be provided (shown ore clearly in FIGS. 3-4). The seal 60 is described in more detail below.

FIG. 3 illustrates a cross section of the connection system as it may be used to secure the catheter 50 to the port 40. As clearly shown in this view, the restraint 20 may include the first interlocking surface 90 located at a proximal end of the restraint. The first interlocking surface 90 may be configured to secure the catheter 50 to the pump 300, e.g., at port 40, by engaging the second interlocking surface 80 associated with the port.

In certain embodiments, at least a portion of the proximal end of the restraint 20 (including the first interlocking surface 90) may be compressible (e.g., deformable) in a radially inward direction, such that the first interlocking surface may engage the second interlocking surface 80 when the proximal end of the restraint is so compressed. For example, the first interlocking surface 90 may include one or more inwardly extending protrusions capable of substantially engaging the second interlocking surface 80 when compression is applied to the proximal end of the restraint. Alternatively, the one or more protrusions may substantially engage the second interlocking surface so that there is substantially no sustained compression on the proximal end of the restraint. Such an alternative embodiment may relieve stress on the restraint 20 and/or the extension members 120, which may be desirable for, e.g., long-term implantation.

In one embodiment, the first interlocking surface 90 is formed by one or more circumferential, inwardly extending protrusions, and the second interlocking surface is defined by a corresponding circumferential groove formed on an external surface of the port 40. However, the first interlocking surface 90 and the second interlocking surface 80 may be shaped in any manner that permits effective engagement therebetween. Preferably, effective engagement is achieved without substantial compression applied to the restraint 20. For example, other embodiments of the present invention could include a first interlocking surface located at the proximal end of the restraint that is defined by an indentation, and a second interlocking surface associated with the port of the medical device that is defined by a protrusion substantially complementary thereto. In still other embodiments, the first interlocking surface may be configured to grippingly engage the second interlocking surface when the proximal end of the restraint 20 is radially compressed.

An optional locking sleeve 10 may be provided and configured to surround the restraint 20 and may be used to assist in securing the proximal end of the restraint relative to the pump 300 by, e.g., providing compression to the extension members 120 located at the proximal end of the restraint, or by preventing expansion of the extension members 120. In certain embodiments, the proximal end of the restraint 20 may be expandable such that, upon relief of a compressing force applied to the proximal end, the proximal end of the restraint 20 may expand substantially to its pre-compressed dimensions. The proximal end of the restraint 20 may be self-expandable. Alternatively, the proximal end of the restraint 20 may be expanded by applying a radially outward force to the restraint 20, preferably applied at the proximal end.

Alternatively, the first interlocking surface 90 of the restraint 20 may be configured to engage the second interlocking surface 80 associated with the port 40 of the pump 300 while in a relaxed state, i.e., without application of a radially compressive force such as that which may be provided by the locking sleeve 10. Thus, the first interlocking surface 90 may engage the second interlocking surface 80 by expanding in a radially outward direction and sliding over the port 40 and into engagement with the second interlocking surface 80. The restraint 20, in particular the proximal end of the restraint 20 including the first interlocking surface 90, preferably has sufficient resiliency to return substantially to the pre-expanded dimensions upon release of the radially outward expanding force imparted, e.g., by sliding the restraint 20 over the seal 60 and port 40.

In some embodiments, the locking sleeve 10 may be secured relative to the restraint 20 when placed in the fully installed (e.g., locked) position. For example, the locking sleeve 10 may interlock with the restraint 20 when the locking sleeve 10 is in an installed position as shown in FIGS. 3 and 4. To permit such interlocking, one or more tabs 70 (e.g., two tabs as show in FIG. 2A) may be provided at or near a distal end of an exterior surface of the restraint 20. The tabs 70 may be configured to secure the locking sleeve 10 relative to the restraint 20 when the locking sleeve is placed in its desired installed position over the restraint 20. In some embodiments, the tabs 70 extend in a radial direction sufficiently to form a groove for receiving a portion of the locking sleeve 10 when the latter is in the desired installed position, as shown in FIGS. 3 and 4. That is, a portion of the locking sleeve 10 near the opening 600 (see FIG. 1A) may have a smaller diameter than the tab 70, and may have a length 800 (see FIG. 3) less than or equal to a length 810 of the groove formed by the tab 70, thereby allowing the restraint 20 to retain the locking sleeve 10 within a defined range of relative longitudinal positions. The locking sleeve 10 may include a protrusion 910, e.g., a circumferential ridge, to facilitate sliding of the locking sleeve relative to the restraint 20.

Thus, the locking sleeve 10 may be secured relative to the restraint 20 by the longitudinal capture of at least a portion of the locking sleeve 10 within the groove formed by the tab 70. As a result, the sleeve 10 may be secured relative to the restraint 20 with a small compressive force or, preferably, substantially without a compressive force.

The tabs 70 may further provide a stop at the distal end of the restraint 20 to assist in preventing the locking sleeve 10 from inadvertently sliding off the restraint in the distal direction. In certain embodiments, the distal end of the restraint 20, e.g., including the tabs 70, is deformable (e.g., compressible) in a radially inward direction, allowing the locking sleeve 10 to selectively slide over the restraint 20 for removal. In other embodiments, the restraint 20 and/or the locking sleeve 10 may temporarily deform under pressure to permit relative movement past the tabs 70.

In certain embodiments, the distal portion of the restraint 20 (e.g., the tabs 70), may be biased outwardly to facilitate engagement of the locking sleeve 10 within the groove formed by the tabs 70. That is, the distal portion of the restraint 20 may be expandable such that, upon relief of any radially inward compressing force (such as that applied by the locking sleeve 10 as the latter is installed), the distal portion of the restraint may expand substantially to its pre-compressed dimensions. In one embodiment, the distal portion of the restraint 20 is self-expandable.

As illustrated in FIG. 3, the locking sleeve 10 may be configured to slide over the distal portion of the restraint 20 in a linear manner. Alternatively, as further described below, the locking sleeve 10 may be configured to engage the restraint 20 in a helical or screw-like manner. In the case of the latter, the locking sleeve 10 may compress the distal portion of the restraint 20 as the locking sleeve is attached. The distal portion of the restraint 20, again, may be configured to expand substantially to its pre-compressed dimensions after such a locking sleeve 10 moves beyond the tab 70, such that the tab secures the locking sleeve in a predetermined position relative to the restraint. The size and shape of the tab 70 and corresponding groove may be selected based on a variety of factors including, but not limited to, ease of use and potential damage to the catheter.

In other embodiments, the locking sleeve 10 may include an expandable portion configured to expand in a radially outward direction upon engagement with the groove, as described below. If the locking sleeve 10 includes such an expandable portion, the expandable portion is preferably contractible such that contraction of the expandable portion of the locking sleeve prevents the locking sleeve from sliding distally off the restraint 20. As a result, the locking sleeve 10 may be secured at a position relative to the restraint 20. Of course, a distal portion of the restraint 20 may be expandable, and a portion of the locking sleeve 10 may be contractable.

addition to engaging the port 40 of the pump 300, the restraint 20 may further operably engage the catheter 50 to secure it relative the pump. For example, the restraint 20, in one embodiment, may include a mating element 140 configured to engage a flanged seal element 150 of a seal 60 of the catheter 50, discussed in further detail below. The mating element 140 and the flanged seal element 150 may be shaped in a manner such that the mating element engages the flanged seal element at least by a contact portion 145 located on an interior surface of the restraint. For example, in the embodiment illustrated in FIG. 4, the contact portion is configured to abut an annular face 155 of the flanged seal element 150 to secure the restraint 20 relative to the catheter 50. The term “abut,” and derivations thereof, are understood herein to mean “in contact with,” “to bring into contact with,” etc., wherein the contact occurs at least at one point between the surfaces having the abutting relationship. In this embodiment, the annular face 155 is substantially normal to a longitudinal axis of the catheter 50.

To abut one another as described above, the mating element 140 of the restraint 20 may form an indentation or recess 165 as shown in FIG. 4, while the flanged seal element 150 may include a corresponding flange or protrusion extending into the indentation.

As discussed further below, the seal 60 may further include a sealing portion, which, in one embodiment, is a tapered sealing portion. The sealing portion may have a shape substantially similar to an inner surface of the locking sleeve 10 as shown in FIG. 4. That is, the restraint 20 may include an opening at its distal end having a diameter similar to a portion of the tapered sealing portion of seal 60, such that the restraint may engage the seal. Further, the sealing portion and the restraint 20 may provide therebetween a small space or recess 167, as shown in FIG. 3, such that there is little or substantially no contact between the restraint 20 and the sealing portion when the connection system is in a locked position.

The locking sleeve 10, in certain embodiments, may include the opening 600 (see FIG. 1A) having a diameter smaller than a shoulder 500 of the restraint 20, preventing the locking sleeve from moving too far proximally relative to the restraint. Additionally, the opening 600 may have a diameter less than the outer diameter formed by the distal end of the restraint 20 (e.g., the tabs 70) such that, after the locking sleeve 10 is slid over the restraint 20 and past the tab 70, the tab 70 prevents the locking sleeve from sliding off the restraint in the distal direction.

In some embodiments, the restraint 20 and the locking sleeve 10 may be provided as a single piece. In other embodiments as illustrated herein, the restraint 20 and the locking sleeve 10 may be provided as separate pieces and, further, may individually include one or more additional components.

A connection system according to various embodiments of the present invention may be provided to couple the catheter 50 to the port 40 of a medical device, such as an implantable infusion pump, through any known means. For example, a physician may use the connection system described herein to secure a catheter to a pump by hand. Alternatively, tools may also be used, as desired by the physician, during the connection process. Preferably, however, connection systems in accordance with many embodiments of the present invention do not require the use of tools to connect the catheter to the medical device port. However, if tools are used, the tools are preferably those standard tools that can be found in most any surgical environment.

Most any biocompatible and biostable material, or combinations thereof, capable of performing the desired securing function may be used to make the exemplary components of the connector systems described and illustrated herein. Nonlimiting examples of suitable material include titanium, polycarbonate, polysulfone (e.g., P1700, available from Solvay Advanced Polymers, and GAFONE, available from Gharda Chemicals, Ltd.), nylon (e.g., GRILAMID nylon 12 and GRILAMID TR 55, available from EMS-Grivory), polyurethane (e.g., polyurethane 75D and TECOTHANE TT-1075DM, available from the Thermedics division of Viasys Healthcare), polyetheretherketone, and polypropylene. The locking sleeve 10 and restraint 20 may be made of the same or different material. For example, the locking sleeve 10 may be made of titanium and the restraint 20 may be made of polysulfone.

In some embodiments, the restraint 20 and the locking sleeve 10 may be different colors. Different colored restraints and locking sleeves may be desirable for many reasons. For example, when a restraint 20 engages a locking sleeve 10, the locking sleeve 10 may be more readably identifiable when the restraint is of a different color than the locking sleeve. A different colored restraint 20 may also be beneficial to facilitate disconnecting the catheter 50 from the port 40.

Any coloring agent may be used to color the locking sleeve 10 and the restraint 20. However, biocompatibility and the intended duration of the implant are preferably considered when choosing coloring agents. For example, some coloring agents are currently deemed suitable for short-term implantation, while others are more suited for more long-term applications. Examples of biocompatible coloring agents suitable for long term implant include TiO₂ and carbon black.

While not wishing to limit its application, a connection system according to an embodiment of the present invention may be applicable with a variety of implantable pumps, including Medtronic's model SynchroMed EL, SynchroMed II, and IsoMed pumps. Preferably, connection systems in accordance with embodiments of the present invention may resist accidental disconnection of the catheter from the pump after implantation. Further, it is preferred that connection systems in accordance with the present invention are readily disconnectable after implantation, even after fibrosis.

B. Catheter and Seal

Connection systems in accordance with embodiments of the present invention may include a catheter assembly or catheter 50 as illustrated in FIGS. 3 and 4. The catheter 50 may include a piece of medical tubing, e.g., tubular member 170, having a distal portion and a proximal portion, wherein the proximal portion is configured for connection to the port 40. The catheter 50 may further include a seal 60, which, in some embodiments, is molded to the proximal end of the tubular member 170. Alternatively, the seal 60 may be attached to the catheter 50, by other known methods, such as with an adhesive, ultrasonic welding, etc.

The seal 60 is preferably disposed about the proximal portion of the tubular member 170 of the catheter 50. The seal 60 preferably includes the flanged seal element 150 and the sealing portion, as discussed herein. The flanged seal element 150 defines the annular face 155 at its distal end that is, preferably, substantially normal to the longitudinal axis of the catheter 50. The annular face 155 is preferably configured to abut the contact portion 145 of the restraint 20, as shown in FIG. 4 and as described in further detail above. Additionally, a proximal end of the flanged seal element 150 may form the proximal end of the catheter 50. In some embodiments, the proximal end or face of the flanged seal element 150 is coextensive with the end of the medical tubing 170, while in other embodiments, the proximal end of the flanged seal element 150 extends beyond the proximal end of the catheter, as illustrated in FIGS. 3 and 4. Without being held to any particular theory, it is believed that such extension of the seal beyond the tubular member 170 may assist in the formation of the well-sealed connection provided by systems in accordance with embodiments of the present invention.

The seal 60 may further include a sealing portion that surrounds a portion of the tubular member 170 and extends distally from the annular face 155 of the flanged seal element 150. The sealing portion may be integral with the flanged seal element 150 (e.g., molded thereto) or, alternatively, may be attached by adhesive, ultrasonic welding, etc. Further, the sealing portion may have any desired shape, provided the contact portion 145 of the restraint 20 is permitted to abut the annular face 155 of the flanged seal element 150 as shown in FIGS. 3 and 4.

Preferably, the sealing portion includes an outer surface that is tapered. In one embodiment, the restraint 20 may contact the sealing portion of the seal 60 at least at one location along an exterior surface of the sealing portion when a separating force is applied between the catheter 50 and the port 40 (FIG. 4). Further, the sealing portion may be configured such that there is substantially no contact between the restraint 20 and the sealing portion (e.g., a space or recess 167 is provided, FIG. 3) in the absence of a longitudinal outside force applied to the connection system (e.g., the system is in a relaxed state). Under either of the foregoing conditions, an adequate connection is provided between the catheter 50 and the port 40.

The seal 60 may be made of any material suitable for forming a fluid barrier, and the flanged seal element and the sealing portions of the seal may be made of the same or of different material. For example, the entire seal, or a portion thereof, may be made of silicone.

C. Connection System

The systems described herein may include the catheter 50 and some or all of the connection system elements described above. For example, the system may include the catheter 50 and the restraint 20 configured to secure the proximal end of the catheter relative to the port 40 of the implantable pump 300. The connection system may further include the locking sleeve 10 to assist in securing the restraint 20 in a position relative to the pump 300 and to the catheter 50. Examples of the connection system, catheter 50, restraint 20, and locking sleeve 10 may be any connection system, catheter 50, restraint 20, and locking sleeve 10 described herein. The system may further include the pump 300 having the port 40 to which the catheter 50 may be secured via the connection system. The port 40 itself may include a port feature, e.g., the second interlocking surface 80. The restraint 20 may include a feature, e.g., the first interlocking surface 90, complementary to the port feature, wherein the first interlocking surface is configured to engage the second interlocking surface to secure the restraint 20 to the pump 300.

D. Methods

Various methods for coupling a catheter 50 to a port 40 of a medical device are contemplated. Such methods provide, among other benefits, relief of stress placed on a catheter that is coupled to the port. Based on the disclosure herein, many such methods will be readily evident to one skilled in the art.

By way of example, an embodiment of the invention provides a method for relieving stress on a catheter coupled to a port of a medical device. FIGS. 1A-1C illustrate an exemplary method in accordance with one embodiment of the present invention. The exemplary method may include coupling the port 40 to a proximal end of the catheter 50 as shown in FIG. 1B, wherein the proximal end of the catheter includes a flanged seal element 150 (see FIGS. 3 and 4) that defines the annular face 155 that is substantially normal to a longitudinal axis of the catheter. The flanged seal element 150 may define an opening for receiving the port 40 of the medical device, wherein the opening is in fluid communication with a lumen of the catheter 50 (see FIG. 3).

The method further includes sliding a tubular restraint 20 over the flanged seal element 150 (see FIG. 1B), the restraint 20 including a contact portion 145 located on an interior surface of the restraint 20 (see FIGS. 3 and 4), wherein the contact portion is operable to abut the annular face 155 of the flanged seal element. The restraint 20 is joined to the port 40 of the medical device by the first interlocking surface 90 (located on the interior surface at or near the proximal end of the restraint 20) as it engages the second interlocking surface 80 located on, or otherwise associated with, the port 40 of the pump 300. In certain embodiments of the present invention, engaging the first interlocking surface 90 of the restraint 20 with the second interlocking surface 80 includes engaging a first inwardly extending radial protrusion of the restraint 20 with an external groove formed on the port 40.

Preferably, with some embodiments of the present invention, the seal 60 and restraint 20 are selected such that the outer dimension of the flanged seal element is smaller than a corresponding portion of the interior surface of the restraint, thus providing the ability to freely rotate the restraint about the seal when the restraint is placed over the seal and the first interlocking surface 90 is engaged with the second interlocking surface 80.

A method of the present invention may further include applying a separating force between the catheter 50 and the port 40. The contact portion 145 located at the interior surface of the restraint 20 thereby abuts the annular face 155 of the flanged seal element 150 at the distal end of the seal element as shown in FIG. 4.

In further embodiments, a locking sleeve 10 may be slid over the restraint 20 (see FIG. 1C), wherein the locking sleeve may be secured by the tab 70 on an exterior surface of the restraint at or near its distal end. In such a configuration, the locking sleeve 10 and restraint 20 may be freely rotated, either individually or together as a single unit, about the seal 60.

Various methods for releasing the catheter 50 from the pump 300 are also contemplated. Based on the disclosure herein, many such methods will be readily evident to one skilled in the art. By way of example, an embodiment of the invention provides a method for releasing the catheter 50 from the pump 300, the method including depressing the distal end of the restraint 20 including the tab 70 engaging the locking sleeve 10. The method further includes sliding the locking sleeve 10 over the depressed end of the restraint 20 to remove the locking sleeve 10 from the restraint 20. The method further includes uncoupling the restraint 20 and catheter 50 from the pump 300.

### II. EXAMPLES

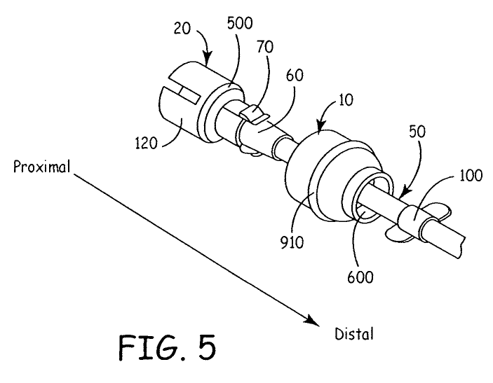

In addition to the embodiments described in detail above and illustrated in FIGS. 1A-1C, 2A-2B, 3, and 4, other embodiments are also contemplated. For example, FIG. 5 illustrates a connection system substantially similar to the systems already described above. For example, the system includes a catheter 50, a seal 60, a restraint 20, and a locking sleeve 10 that are designed to operate in a manner substantially similar to that already described above. However, the embodiment of FIG. 5 may further incorporate a stopping mechanism 100. The stopping mechanism 100 may be configured to prevent the locking sleeve 10 and/or the restraint 20 from sliding off, or excessively towards, the distal end of catheter 50. The stopping mechanism 100, or a portion thereof, may have a diameter greater than the distal opening of the restraint 20 and the opening 600 of the locking sleeve 10.

FIGS. 6A and 6B illustrate a connection system in accordance with another embodiment of the invention. This particular embodiment may, once again, include a catheter 50 connected to a port 40 of a pump 300 (not shown) by a sliding type ring connector. FIG. 6A illustrates the system in an unlocked position, while FIG. 6B shows it in a locked position. Like the systems already described herein, the system may include a restraint 20 having a proximal end and a distal end, a locking sleeve 10, and a seal 60 associated with a proximal end of the catheter 50. The proximal end of restraint 20 may include a plurality of extension members 120. The proximal end of restraint 20, including the extension members 120, may be configured to engage the port 40 in a manner similar to that already described herein. The restraint 20 may be configured such that it slides along the catheter 50, and locking sleeve 10 may similarly slide along the restraint. By sliding the locking sleeve 10 in the proximal direction, the locking sleeve 10 may move beyond one or more tabs 70. The proximal end of the restraint 20 (including the tabs 70) may be compressed radially inward as locking sleeve 10 slides over the restraint 20. Once the locking sleeve 10 is in the desired locking position, the tabs 70 may return to their natural position, thereby locking the restraint in the installed position. Instead of (or in addition to) compressing the restraint 20 (e.g., the tabs 70), the locking sleeve 10 could be expanded radially outward as the locking sleeve is slid over the restraint and then return to its pre-expanded position once it passes the tabs 70.

After the locking sleeve 10 is slid over the tab 70, the restraint 20 may expand (or, as mentioned above, the locking sleeve 10 may contract) such that the locking sleeve is held in the desired longitudinal position relative to the restraint by the tab. The locking sleeve 10, it turn, may assist in ensuring that the restraint 20 remains in a substantially fixed longitudinal position relative to the port 40. The restraint 20 may further include a protrusion 510 to ensure that the locking sleeve 10 does not move too far in the proximal direction (e.g., the protrusion 510 may have an outer diameter greater than the diameter of an opening on the proximal end of the locking sleeve 10).

To remove the locking sleeve 10 from the restraint 20, the restraint may be depressed (e.g., at the tab(s) 70 and/or the extension member(s) 120) to allow the locking sleeve to slide in the distal direction.

The catheter 50 illustrated in FIGS. 6A and 6B may also include a seal 60 disposed about the proximal end of the catheter 50. The seal 60 may be configured substantially as already described herein, e.g., may have a tapered sealing portion, having its smallest diameter distally in the taper and its largest diameter proximally in the taper.

The distal portion of restraint 20 may also include an opening having a diameter. The restraint 20 may be slid in a proximal direction over the catheter 50 until the opening of restraint 20 engages the tapered sealing portion of the seal 60 having a diameter substantially similar to the diameter of the opening. Locking the restraint 20 relative to the port 40, may thereby secure the catheter 50 relative to the pump 300 (not shown) as already described herein.

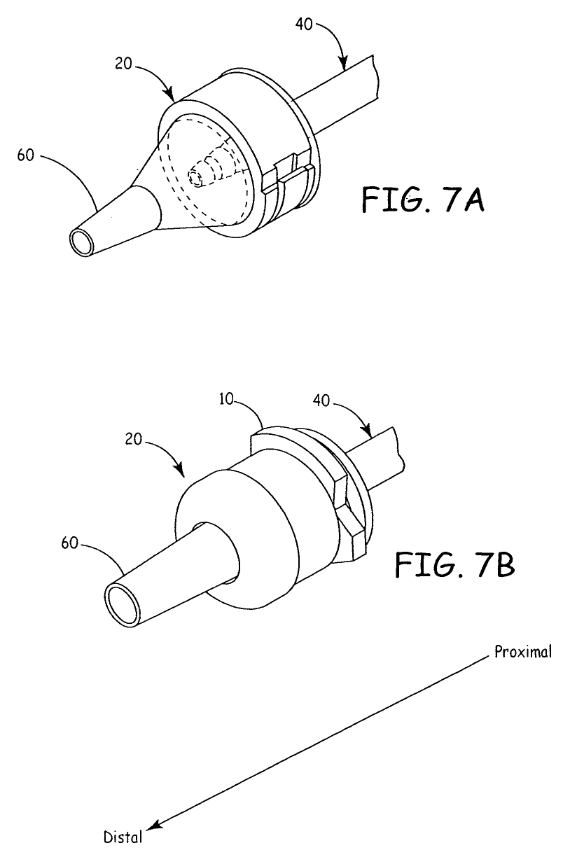

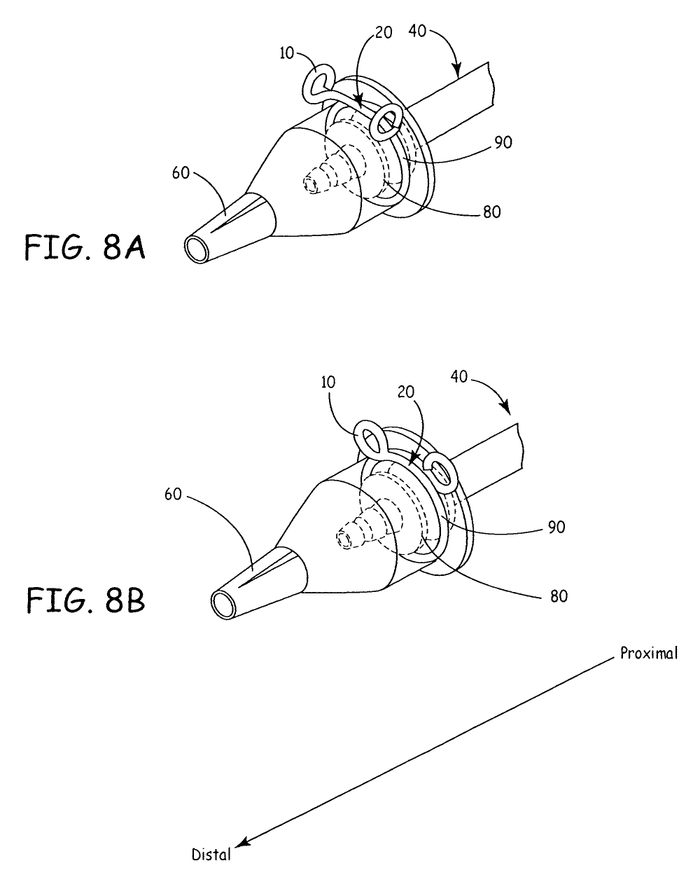

FIGS. 7A and 7B show a seal 60 of a catheter 50 (not shown) connected to a port 40 of a pump 300 (also not shown) by a snap type connector including a restraint 20 and a locking sleeve 10. Similarly, FIGS. 8A and 8B show a seal 60 of a catheter 50 (not shown) connected to a port 40 of a pump 300 (also not shown) by a compression or snap ring type connector also having a restraint 20 and a locking sleeve 10. FIGS. 7A and 8A show an unlocked position of the respective connection system, while FIGS. 7B and 8B show the respective system in a locked position.

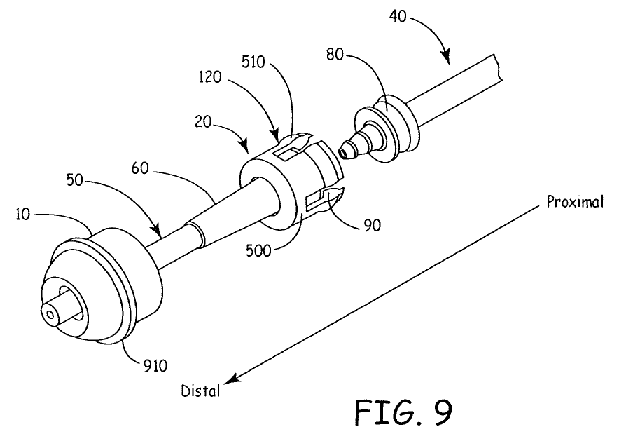

FIG. 9 illustrates another embodiment of the invention utilizing a sliding ring type connector. Once again, the system of FIG. 9 includes a catheter 50 having a seal 60 with a tapered sealing portion as already discussed herein with respect to FIGS. 1-4. A restraint 20 is also provided that includes an opening through its distal and proximal ends. The opening may have a diameter that is similar to at least a portion of the tapered sealing portion of the seal 60. The restraint 20 may also include a plurality of extension members 120 configured to engage a port 40 of a pump 300 (not shown). The port 40 may includes a second interlocking surface 80, e.g., circumferential groove. The extension members 120 further include a first interlocking surface 90, e.g., a plurality of inwardly extending protrusions and, optionally, an outwardly extending protrusion 510. As described elsewhere herein, the first interlocking surface 90 may selectively engage the second interlocking surface 80 of port 40 to interlock the restraint with the port. Such interlocking of the restraint 20 may occur either when the restraint is compressed (e.g., with a locking sleeve 10) or when in a relaxed state.

The locking sleeve 10 shown in FIG. 9 may include a distal opening having a diameter. The diameter of the distal opening the locking sleeve may be less than the outer diameter of the shoulder 500 of the restraint 20, such that when the locking sleeve 10 is slid over the restraint into a locked position, the locking sleeve does not slide too far proximally.

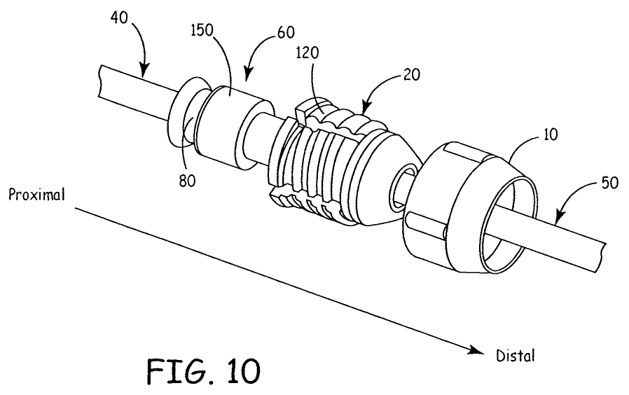

FIG. 10 illustrates an embodiment of a screw-on lock type connection system. The system may, once again, include a catheter 50 for coupling with a port 40, a seal 60 associated with the catheter 50, a restraint 20, and a locking sleeve 10. The restraint 20 may include one or more helical grooves on its exterior surface. The locking sleeve 10 may include complementary protrusions or threads (not shown) that allow the locking sleeve to be screwed into a secured position about the restraint 20. As the locking sleeve 10 is tightened over the restraint, it may compress the extension members 120, causing first interlocking surfaces (not shown) of the restraint to engage a second interlocking surface 80 of the port 40 as already described herein. Otherwise, the connection system illustrated in FIG. 10 may be used to secure the catheter 50 to the port 40 in a similar manner to the previously described embodiments.

Connection systems in accordance with embodiments of the present invention provide numerous advantages over know catheter attachment systems. For example, systems in accordance with the present invention provide connection and sealing of a catheter to a port of a medical device without sutures. Further, connection systems as described herein are configured to relieve stress on the catheter itself, thereby improving the durability and integrity of the overall connection. By incorporating the elements described herein, connection systems in accordance with embodiments of the present invention may provide a well-sealed connection between the catheter and the port substantially, and preferably solely, through longitudinal interlocking of a restraint as described herein with both the port and the catheter. Thus, restraints 20 of the present invention, either with or without the described locking sleeve 10, may be rotatable about the seal of the catheter (e.g., about the flanged seal element) when the first interlocking surface 90 is engaged with the second interlocking surface 80.

Various embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow, and equivalents thereto.

1. A connection system for coupling a catheter to a port of a medical device, the system comprising:
a seal attached at or near a proximal end of the catheter, the seal comprising a flanged seal element defining an annular face that is substantially normal to a longitudinal axis of the catheter; and
a tubular restraint configured to slide partially or completely over the seal, wherein the restraint comprises:
a contact portion located on an interior surface of the restraint, the contact portion configured to abut the annular face of the flanged seal element; and
a first interlocking surface formed on the interior surface at or near a proximal end the restraint, the first interlocking surface configured to engage a second interlocking surface associated with the port of the medical device, wherein engagement of the first interlocking surface with the second interlocking surface substantially secures the catheter relative to the port.

2. The connection system of claim 1, wherein the first interlocking surface comprises an inwardly extending protrusion, and the second interlocking surface comprises a circumferential groove formed on an external surface the port.

3. The connection system of claim 1, wherein the seal further comprises a sealing portion extending distally from the flanged seal element.

4. The connection system of claim 3, wherein the sealing portion comprises a tapered outer surface.

5. The connection system of claim 1, wherein the seal is attached to the catheter with an adhesive.

6. The connection system of claim 1, wherein the seal is ultrasonically welded to the catheter.

7. The connection system of claim 1, wherein the seal is molded to the catheter.

8. The connection system of claim 1, wherein the seal extends beyond the proximal end of the catheter.

9. The connection system of claim 1, further comprising a tab located on an exterior surface of the restraint at or near a distal end of the restraint.

10. The connection system of claim 9, wherein the distal end of the restraint is deformable.

11. The connection system of claim 1, further comprising a locking sleeve to surround the restraint.

12. The connection system of claim 11, wherein the locking sleeve interlocks with the restraint when the locking sleeve is in an installed position.

13. The connection system of claim 1, wherein the restraint is rotatable about the seal when the first interlocking surface is engaged with the second interlocking surface.

14. The connection system of claim 1, wherein the medical device comprises an implantable infusion pump.

15. A method for relieving stress on a catheter coupled to a port of a medical device, the method comprising:
coupling the port to a proximal end of the catheter, wherein the proximal end comprises a flanged seal element defining an annular face substantially normal to a longitudinal axis of the catheter, and further wherein the flanged seal element defines an opening for receiving the port, the opening in fluid communication with a lumen of the catheter;
sliding a tubular restraint over the flanged seal element, wherein the restraint comprises: a contact portion located on an interior surface of the restraint, the contact portion operable to abut the annular face of the flanged seal element; and a first interlocking surface located on the interior surface at or near a proximal end of the restraint; and
engaging the first interlocking surface of the restraint with a second interlocking surface located on the port.

16. The method of claim 15, further comprising selecting the outer dimension of the flanged seal element so that it is smaller than a corresponding portion of the interior surface of the restraint.

17. The method of claim 15, further comprising:
applying a separating force between the catheter and the port; and
abutting the contact portion of the restraint against the annular face of the flanged seal element.

18. The method of claim 15, further comprising sliding a locking sleeve over the restraint.

19. The method of claim 18, further comprising securing the locking sleeve with a tab formed at or near a distal end of an exterior surface of the restraint.

20. The method of claim 19, further comprising rotating the locking sleeve and the restraint about the seal.

21. The method of claim 15, wherein engaging the first interlocking surface of the restraint with the second interlocking surface comprises engaging a first inwardly extending radial protrusion of the restraint with an external groove formed on the port.

22. A connection system for coupling medical tubing to a port of a medical device, the system comprising:
a catheter assembly comprising the medical tubing and a seal attached at or near a proximal end of the medical tubing, the seal comprising: a flanged seal element defining an annular face that is substantially normal to a longitudinal axis of the medical tubing; and a tapered sealing portion extending distally from the annular face of the flanged seal element;
a tubular restraint configured to slide partially or completely over the seal, wherein the restraint comprises:
a contact portion located on an interior surface of the restraint, the contact portion configured to abut the annular face of the flanged seal element; and
a first interlocking surface formed on the interior surface at or near a proximal end the restraint, the first interlocking surface configured to engage a second interlocking surface associated with the port of the medical device, wherein engagement of the first interlocking surface with the second interlocking surface substantially secures the medical tubing relative to the port; and
a locking sleeve configured to surround the restraint, wherein the locking sleeve is securable relative to the restraint.

23. The system of claim 22, further comprising the medical device.

24. The system of claim 23, wherein the medical device is an implantable infusion pump.
